(19) United States
(12) Patent Application Publication
(10) Pub. No.: US 2024/0066511 A1
(43) Pub. Date: Feb. 29, 2024

(54) MN-MOF-BASED COLD-ADAPTED NANOZYME AND PREPARATION METHOD THEREOF

(71) Applicant: NORTHWESTERN POLYTECHNICAL UNIVERSITY, Xi'an City (CN)

(72) Inventors: Yao CHEN, Xi'an City (CN); Lianbing ZHANG, Xi'an City (CN); Yong QIN, Xi'an City (CN); Qing TIAN, Xi'an City (CN); Ruiting HAN, Xi'an City, (CN); Luqiu YANG, Xi'an City (CN)

(21) Appl. No.: 18/500,939

(22) Filed: Nov. 2, 2023

Related U.S. Application Data
(63) Continuation of application No. PCT/CN2021/096152, filed on May 26, 2021.

(30) Foreign Application Priority Data
May 11, 2021 (CN) 202110513002.0

(51) Int. Cl. B01J 31/16; B01J 35/02; B01J 37/00

(52) U.S. Cl. CPC B01J 31/1691; B01J 35/023; B01J 37/0072; B01J 2531/72; B82Y 5/00

(57) ABSTRACT

A preparation method of a Mn-MOF-based cold-adapted nanozyme and a preparation method thereof. The method includes: preparing a Mn(CH₃COO)₃·2H₂O solution by fully dissolving a manganese-containing precursor Mn(CH₃COO)₃·2H₂O in a mixed solution of an alcohol and distilled water; wherein the alcohol is ethanol or methanol; preparing a benzenetricarboxylic acid solution by fully dissolving benzenetricarboxylic acid solid in the mixed solution of the alcohol and distilled water; reacting the Mn(CH₃COO)₃·2H₂O solution with the benzenetricarboxylic acid solution sufficiently using co-precipitation; and centrifuging and removing a supernatant to obtain the MnBTC, where the MnBTC is nano-metal-organic framework (nano-MOF) with a particle size of less than 10 nm.

### CROSS REFERENCE TO RELATED APPLICATION

The present disclosure is a continuation-application of International (PCT) Patent Application No. PCT/CN2021/096152, filed on May 26, 2021, which claims priority of Chinese Patent Applications No. 202110513002.0, filed on May 11, 2021, the entire contents of which are hereby incorporated by reference in their entirety.

### TECHNICAL FIELD

The present disclosure relates to the fields of nanobiology technologies, and in particular to a Mn-MOF-based cold-adapted nanozyme and a preparation method thereof.

### BACKGROUND

More than 80% of the earth's environment is a cryobiosphere (<5° C.), and natural cold-adapted enzymes (also known as cryotolerant enzymes) play an irreplaceable role in maintaining normal biochemical reactions and ecological virtuous cycle in these extreme environments. In modern industrial production, cold-adapted enzymes also have important applications in biomedicine, sewage treatment, food processing and textile industry. However, the cold-adapted enzymes have a major disadvantage of poor thermal stability, and are very easy to be denatured and inactivated with temperature reaching the mesophilic zone. This makes it difficult to realize the batch cloning expression by traditional enzyme engineering, and severely limits their practical application in industrial production.

Nanozyme are inorganic nanomaterials with intrinsic enzyme-like properties. Compared with traditional artificial enzymes, they have many advantages, such as good stability, easy preparation, low cost, easy batch production, excellent activity, and flexible regulation, thus showing extraordinary application prospects in many cutting-edge fields of biomedicine. Currently, more than 300 inorganic nanomaterials have been reported to possess enzyme activities such as peroxidase, oxidase, catalase, superoxide dismutase, etc., but there is no report on the mimicking of the natural cold-adapted enzymes. Therefore, the development of efficient and stable cold-adapted nanozyme is still a major challenge for current research, which is expected to overcome the limitations of traditional enzyme engineering and should open up a new horizon for the application of cold-adapted enzymes.

### SUMMARY OF THE DISCLOSURE

The purpose of the present disclosure is to provide a Mn-MOF-based cold-adapted nanozyme and a preparation method thereof, which can be used to replace cold-adapted enzymes which are difficult to be extracted and isolated in nature and have very poor stability. The proposed cold-adapted nanozyme can be applied in the fields of biomedical engineering, ecological and environmental governance, etc. in extreme environments.

The present disclosure is specifically implemented by the following technical solutions.

A first aspect of the present disclosure provides a Mn-MOF nanoparticle for use as a natural cold-adapted enzyme mimic, the Mn-MOF being a nano-MOF with a particle size of less than 10 nm.

In some embodiments, the Mn-MOF nanoparticle is MnBTC or nano MIL-100(Mn).

A second aspect of the present disclosure provides a preparation method of the MIL-100(Mn) nanoparticle, including:
sufficiently dissolving a manganese-containing precursor Mn(NO₃)₂·4H₂O in methanol to prepare a Mn(NO₃)₂·4H₂O solution;
sufficiently dissolving benzenetricarboxylic acid solid in methanol to prepare a benzenetricarboxylic acid solution;
mixing the Mn(NO₃)₂·4H₂O solution and the benzenetricarboxylic acid solution in a reactor and reacting fully with a hydrothermal method; and
centrifugating to remove a supernatant to obtain the nano MIL-100(Mn).

In some embodiments, a concentration of the Mn(NO₃)₂·4H₂O solution and a concentration of the benzenetricarboxylic acid solution are each 0.1-2 mM.

In some embodiments, the Mn(NO₃)₂·4H₂O solution and the benzenetricarboxylic acid solution are mixed at a volume ratio of 1:5-5:1, and a condition of the reaction is: 120 min at 90-150° C.

On the basis of the above preparation method of nano MIL-100(Mn), the present disclosure further modulates the type of manganese precursor, the temperature of synthesis, and the type and ratio of solvent, to obtain amorphous MnBTC with more active sites, with more excellent enzyme-like activity and low-temperature resistance properties.

Specifically, a third aspect of the present disclosure provides a preparation method of MnBTC, including:
preparing a Mn(CH₃COO)₃·2H₂O solution by fully dissolving a manganese-containing precursor Mn(CH₃COO)₃·2H₂O in a mixed solution of an alcohol and distilled water; wherein the alcohol is ethanol or methanol;
preparing a benzenetricarboxylic acid solution by fully dissolving benzenetricarboxylic acid solid in the mixed solution of the alcohol and distilled water;
reacting the Mn(CH₃COO)₃·2H₂O solution and the benzenetricarboxylic acid solution sufficiently using co-precipitation; and
centrifuging and removing a supernatant to obtain the MnBTC.

In some embodiments, a concentration of the Mn(CH₃COO)₃·2H₂O solution and a concentration of the benzenetricarboxylic acid solution are each 0.1-2 mM.

In some embodiments, a mixing volume ratio of the Mn(CH₃COO)₃·2H₂O solution and the benzenetricarboxylic acid solution is 1:5-5:1, and a condition of the reaction is: 120 min at 50-150° C.

A fourth aspect of the present disclosure provides a Mn-MOF cold-adapted nanozyme, made by the preparation method as any of the above.

In some embodiments, the Mn-MOF cold-adapted nanozyme is an oxidase-like enzyme adapted to a range of 4-37° C.

### BRIEF DESCRIPTION OF THE DRAWINGS

FIG. 1 is a TEM image of MnBTC obtained from Embodiment 8 of the present disclosure, with a scale bar of 100 nm.

FIG. 2 is a TEM image of nano MIL-100(Mn) obtained from Embodiment 1 of the present disclosure, with a scale bar of 100 nm.

FIG. 3 is an enzyme-catalyzed kinetic plot of an oxidase-like activity of nano MIL-100(Mn) obtained from Embodiment 1 of the present disclosure as a function of temperature.

FIG. 4 is an enzyme-catalyzed kinetic plot of an oxidase-like activity of MnBTC obtained from Embodiment 8 of the present disclosure as a function of temperature.

FIG. 5 is an enzyme-catalyzed kinetic plot of natural horseradish peroxidase activity as a function of temperature.

FIG. 6 is an enzyme-catalyzed kinetic plot of Pt nanozyme activity as a function of temperature.

### DETAILED DESCRIPTION

In order to be able to more clearly understand the above purposes, features, and advantages of the present disclosure, the present disclosure is described in detail below in connection with the accompanying drawings and specific embodiments. It is to be noted that the embodiments and the features in the embodiments of the present disclosure can be combined with each other without conflict.

Unless otherwise defined, all technical and scientific terms used herein have the same meaning as commonly understood by those skilled in the art. Terms used herein in the specification of the present disclosure are intended only for the purpose of describing specific embodiments and are not intended to be limiting of the present disclosure.

### Synthesis of Nano MIL-100(Mn)

### Embodiment 1

A manganese-containing precursor Mn(NO₃)₂·4H₂O is dissolved in 20 mL of methanol and stirred until fully dissolved to formulate a 1 mM solution of Mn(NO₃)₂·4H₂O.

168.11 mg of 1,3,5-benzenetricarboxylic acid (BTC) solid is weighed and dissolved in 20 mL of methanol and stirred until fully dissolved to formulate a 1 mM solution of BTC.

The Mn(NO₃)₂·4H₂O solution and the BTC solution are mixed in a 1:1 volume and stirred thoroughly for 10 minutes.

Subsequently, the mixed solution is transferred to a reactor to react hydrothermally at 90° C. for 120 minutes; after the reaction, a precipitate is collected by centrifuging the reaction solution at high speed; the resulting precipitate is washed three times with methanol, and the washed precipitate is nano MIL-100(Mn).

### Embodiment 2

This embodiment differs from Embodiment 1 in that the Mn(NO₃)₂·4H₂O solution and the BTC solution are mixed in a volume of 1:1 and placed in a hydrothermal reaction system at 150° C. for 120 minutes. The resulting nano-MIL-100(Mn) has a slightly reduced activity compared to Embodiment 1, again with a cold-adapted characteristic. Therefore, it is hypothesized that an appropriate increase in the reaction temperature will reduce the oxidase activity of the nano MIL-100(Mn).

### Embodiment 3

This embodiment differs from Embodiment 1 in that the Mn(NO₃)₂·4H₂O solution and the BTC solution are mixed in a volume of 5:1 and placed in a hydrothermal reaction system at 90° C. for 120 minutes. The resulting nano MIL-100(Mn) has a reduced activity and yield than Embodiment 1.

### Embodiment 4

This embodiment differs from Embodiment 1 in that the Mn(NO₃)₂·4H₂O solution and the BTC solution are mixed in a volume of 1:5 and placed to a hydrothermal reaction system at 90° C. for 120 minutes. The activity of the resulting nano MIL-100(Mn) is not significantly different from the Embodiment 1.

### Embodiment 5

This embodiment differs from Embodiment 1 in that the concentration of the Mn(NO₃)₂·4H₂O solution and the BTC solution is 0.1 mM, and the yield of the resulting nano MIL-100(Mn) is reduced and the activity is not significantly different.

### Embodiment 6

This Embodiment differs from Embodiment 1 in that the concentration of the Mn(NO₃)₂·4H₂O solution and the BTC solution is 2 mM, the yield of the resulting nano MIL-100(Mn) is increased and there is no significant difference in activity. Therefore, it is hypothesized that the concentration of the reactants has a greater effect on the yield and a lesser effect on the activity.

### Embodiment 7

This Embodiment differs from Embodiment 1 in that the reaction time is increased to 180 min and the activity of the resulting nano MIL-100(Mn) is slightly reduced than in Embodiment 1, which may be related to the particle size.

### Synthesis of MnBTC

### Embodiment 8

214.48 mg of Mn(CH₃COO)₃·2H₂O is fully dissolved in 20 mL of a mixture solution of ethanol and distilled water (in volume ratio of 1:1) and stirred until fully dissolved to form a 1 mM solution of Mn(CH₃COO)₃·2H₂O.

168.11 mg of 1,3,5-benzenetricarboxylic acid (BTC) is fully dissolved in 20 mL of a mixture of ethanol and distilled water (in volume ratio of 1:1) to form a 1 mM solution of BTC.

The Mn(CH₃COO)₃·2H₂O solution is mixed with the BTC solution at a volume ratio of 1:1 and stirred thoroughly for 10 minutes to mix well.

The mixed solution is heated at 50° C. with water bath conditions under stirring for 120 min.

The mixed solution is then centrifuged and washed twice with ethanol and once with ultrapure water, then the precipitate of MnBTC is obtained.

### Embodiment 9

This embodiment differs from Embodiment 8 in that the concentration of the Mn(CH₃COO)₃·2H₂O solution is 0.1 mM, and the concentration of the BTC solution is 0.1 mM; the Mn(CH₃COO)₃·2H₂O solution is mixed with the BTC solution in a volume ratio of 1:1.

### Embodiment 10

This embodiment differs from Embodiment 8 in that the concentration of the Mn(CH₃COO)₃·2H₂O solution is 2 mM and the concentration of the BTC solution is 2 mM; the Mn(CH₃COO)₃·2H₂O solution is mixed with the BTC solution in a volume ratio of 1:1. The yield of resulting MnBTC is more than that in Embodiment 8, but the activity is slightly reduced.

### Embodiment 11

This embodiment differs from Embodiment 8 in that the Mn(CH₃COO)₃·2H₂O solution is mixed with the BTC solution in a volume ratio of 5:1. The activity of the resulting MnBTC is slightly reduced compared to Embodiment 8.

### Embodiment 12

This embodiment differs from Embodiment 8 in that the Mn(CH₃COO)₃·2H₂O solution is mixed with the BTC solution in a volume ratio of 1:5. The activity of the resulting MnBTC is slightly reduced compared to Embodiment 8.

### Embodiment 13

This embodiment differs from Embodiment 12 in that the concentration of the Mn(CH₃COO)₃·2H₂O solution is 1 mM and the concentration of the BTC solution is 1 mM; the Mn(CH₃COO)₃·2H₂O solution is mixed with the BTC solution in a volume ratio of 1:1. The mixed solution is heated in an oil bath at 90° C. under stirring for 120 min. The resulting MnBTC is less active than Embodiment 8, but also has good cold-adapted characteristics.

### Embodiment 14

This embodiment differs from Embodiment 13 in that the Mn(CH₃COO)₃·2H₂O solution mixing with the BTC solution is in a volume ratio of 1:1. The mixed solution is heated with stirring in an oil bath at 150° C. for 120 min. The resulting MnBTC is less active than Embodiment 13, but also has good low temperature resistance. Therefore, it is hypothesized that the activity of the synthesized MnBTC is reduced as the synthesis temperature increases.

### Embodiment 15

This embodiment differs from Embodiment 14 in that the concentration of the Mn(CH₃COO)₃·2H₂O solution is 1 mM and the concentration of the BTC solution is 1 mM; the Mn(CH₃COO)₃·2H₂O solution is mixed with the BTC solution in a volume ratio of 1:1. The mixed solution is heated with a water bath under stirring at 50° C. for 180 min. The resulting MnBTC has a reduced activity compared to Embodiment 8, but still has good low temperature resistance.

### Embodiment 16

This Embodiment differs from Embodiment 15 in that the concentration of the Mn(CH₃COO)₃·2H₂O solution is 1 mM and the concentration of the BTC solution is 1 mM; the Mn(CH₃COO)₃·2H₂O solution is mixed with the BTC solution in a volume ratio of 1:1. The mixed solution is heated with a water bath under stirring at 50° C. for 240 min. The resulting MnBTC has reduced activity compared to Embodiment 15, but still has good low temperature resistance.

Morphological diagrams of the MnBTC obtained in Embodiment 8 and the nano MIL-100(Mn) obtained in Embodiment 1 are captured for low temperature resistance evaluation.

As shown in FIG. 1, the MnBTC nanozyme has a particle size of about 5 nm and can provide a larger specific surface area as well as more active sites.

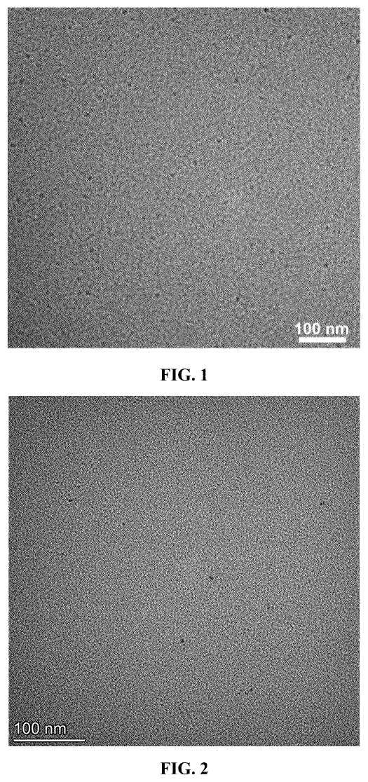

As shown in FIG. 2, the nano MIL-100(Mn) has a particle size of about 8-10 nm and can provide a larger specific surface area as well as more active sites.

I. Evaluation of the Low-Temperature Nanozymatic Performance of Nano MIL-100(Mn) Nanozyme.

10 μL of 3, 3′, 5, 5′-tetramethylbenzidine (TMB, 25 mM) and 8 μL of 2 mg/mL MIL-100(Mn) (dissolved in ethanol) are added to 982 μL of acetic acid-sodium acetate buffer solution (0.2 M, pH 3.6), and kinetic profiles of the reaction are monitored at 4, 20, 30, and 37° C., respectively.

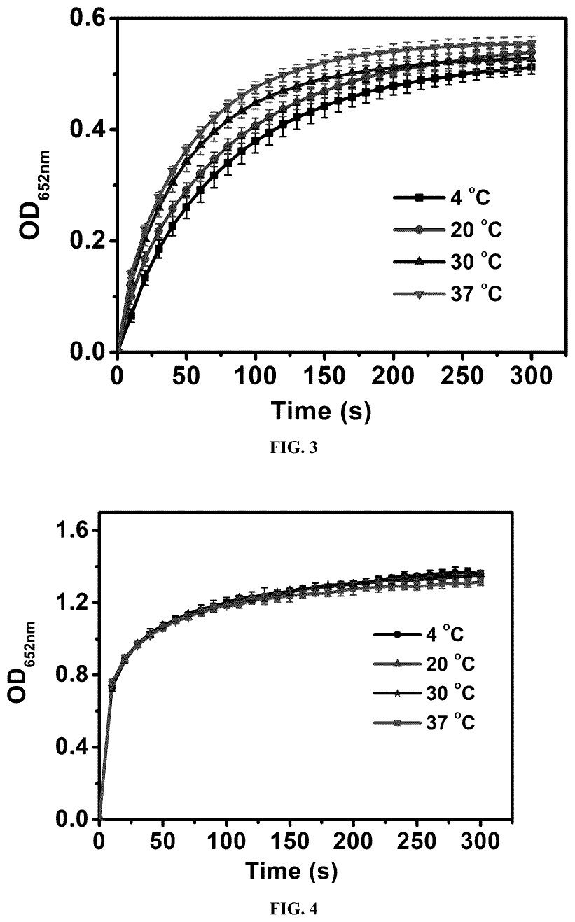

As shown in FIG. 3, lowering the temperature only slightly reduces the initial velocity of the reaction of nano MIL-100(Mn) and the endpoints of the kinetic profiles are consistent, indicating that the nano MIL-100(Mn) nanozyme have good cold-adapted enzymatic-like characteristics.

II. Evaluation of the Low-Temperature Nanozymatic Performance of MnBTC Nanozyme.

The cold-adapted characteristics of MnBTC nanozyme are evaluated in the same manner as nano-MIL-100(Mn), which is performed as follows: 10 μL of 3, 3′, 5, 5′-tetramethylbenzidine (TMB, 25 mM) and 8 μL of 2 mg/mL of MnBTC (dissolved in ethanol) are added to 982 μL of acetic acid-sodium acetate buffer solution (0.2 M, pH 3.6), and kinetic profiles of the reaction system are monitored at 4, 20, 30, and 37° C., respectively.

As shown in FIG. 4, the temperature has almost no effect on the initial velocity of the reaction of MnBTC nanozyme, indicating that MnBTC nanozyme are able to maintain their activity in temperature region from 4 to 37° C., which demonstrates the excellent low-temperature nanozymatic performance of MnBTC.

The MnBTC prepared in Embodiment 8 and the nano MIL-100(Mn) prepared in Embodiment 1 are compared with other natural enzymes and nanozyme with oxidase-like activity as follows.

I. Effect of Temperature on Natural Horseradish Peroxidase Activity.

10 μL of 3, 3′, 5, 5′-tetramethylbenzidine (TMB, 25 mM) and 8 μL of 2 mg/mL horseradish peroxidase are added to 982 μL of acetic acid-sodium acetate buffer solution (0.2 M, pH 3.6), and the enzyme catalytic activity is assayed using a kinetic modeling at 4, 20, 30, and 37° C., respectively, as shown in FIG. 5.

II. Effect of Temperature on the Activity of Pt Nanozyme with Oxidase-Like Activity.

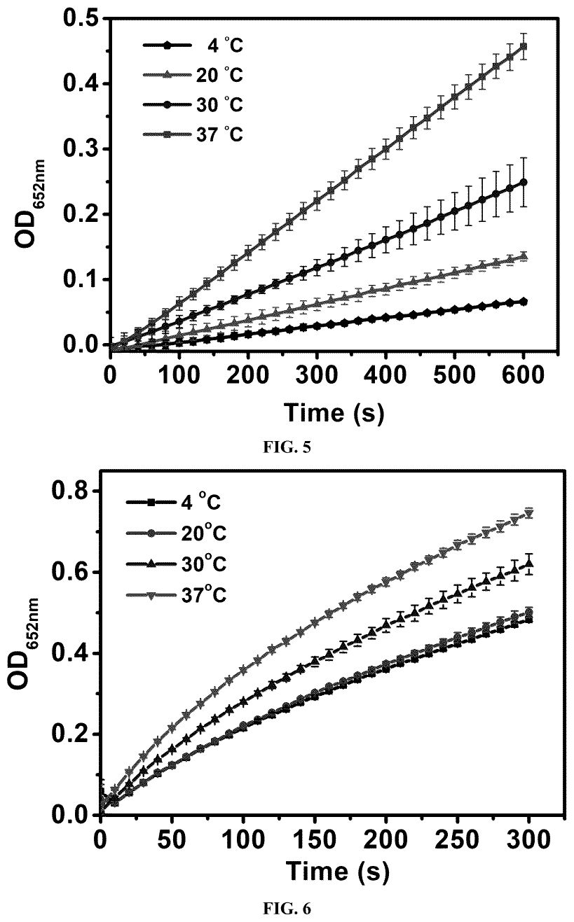

10 μL of 3, 3′, 5, 5′-tetramethylbenzidine (TMB, 25 mM) and 8 μL of 2 mg/mL Pt nanozyme are added to 982 μL of acetic acid-sodium acetate buffer solution (0.2 M, pH 3.6) and the enzyme catalytic activity is assayed using the kinetic modeling at 4, 20, 30, and 37° C., respectively, as shown in FIG. 6.

By FIGS. 5 and 6 compared to nano MIL-100(Mn) in FIG. 3 and MnBTC and in FIG. 4, temperature has a significant effect on the activity of natural horseradish peroxidase and Pt nanozyme. The activity of natural horseradish peroxidase and Pt nanozyme is reduced tremendously with reducing temperature. In contrast, lowering the temperature has only a slight effect or even no effect on the activity of MnBTC and nano MIL-100(Mn), demonstrating that MnBTC and nano MIL-100(Mn) have excellent low-temperature resistance and can be used for oxidative enzyme-catalyzed reaction applications in extremely harsh environments such as low temperatures.

The present disclosure has the following beneficial technical effects compared to the related art.

(1) Both MnBTC and nano MIL-100(Mn) synthesized in the present disclosure have excellent oxidase-like activity at low temperatures, and in the temperature range of 4-37° C., their oxidase-like activity remains constant or undergoes only a slight reduction (<10%) as the temperature reduces.

(2) Both MnBTC and nano MIL-100(Mn) synthesized in the present disclosure have excellent stability and can be stored for a long period of time at room temperature and high temperature.

(3) The present disclosure utilizes Mn(CH₃COO)₃·2H₂O as a metal precursor during the synthesis of MnBTC, which can directly provide trivalent Mn ions and enables the Mn-MOFs with a high proportion of high valence Mn—O bonds (+3 valence, +4 valence) and more active sites, thus endowing them with excellent enzyme-like activities.

(4) The nano-Mn-MOF synthesized in the present disclosure have ultrafine particle sizes, which provides larger specific surface area and enhanced enzyme-like activity.

(5) The preparation method adopted in the present disclosure has simple operating steps, and the reaction conditions are easy to control to achieve batch production easily.

(6) The MnBTC synthesized in the present disclosure is in amorphous form and has more abundant catalytic sites and higher substrate affinity, thus has more excellent enzyme-like activity compared to nano MIL-100(Mn).

Finally, it should be noted that the above-mentioned embodiments are only provided to illustrate the technical solutions of the present disclosure rather than limitations. Although the present disclosure has been described in detail with reference to the preferred embodiments, it should be understood by those skilled in the art that modifications or equivalent substitutions can be made to the technical solutions of the present disclosure without departing from the spirit and scope of the technical solutions of the present disclosure.

What is claimed is:

1. A preparation method for the manganese-based MOF of MnBTC, comprising:
preparing a Mn(CH₃COO)₃·2H₂O solution by fully dissolving a manganese-containing precursor Mn(CH₃COO)₃·2H₂O in a mixed solution of an alcohol and distilled water; wherein the alcohol is ethanol or methanol;
preparing a benzenetricarboxylic acid solution by fully dissolving benzenetricarboxylic acid solid in the mixed solution containing alcohol and distilled water;
mixing the Mn(CH₃COO)₃·2H₂O solution and the benzenetricarboxylic acid solution sufficiently to allow for reaction using co-precipitation method; and the product is centrifuged and the supernatant is removed to obtain the MnBTC; the MnBTC is nanosized metal-organic framework (nano-MOF) with a particle size of less than 10 nm.

2. The preparation method according to claim 1, wherein a concentration of the Mn(CH₃COO)₃·2H₂O solution and a concentration of the benzenetricarboxylic acid solution are each 0.1-2 mM.

3. The preparation method according to claim 1, wherein a mixing volume ratio of the Mn(CH₃COO)₃·2H₂O solution and the benzenetricarboxylic acid solution is 1:5-5:1, and a condition of the reaction is: 120 min at 50-150° C.

4. A Mn-MOF-based cold-adapted nanozyme, made by the preparation method according to claim 1.

5. The Mn-MOF cold-adapted nanozyme according to claim 4, being an oxidase-like enzyme adapted to a range of 4-37° C.
